(19) United States
(12) Patent Application Publication
(10) Pub. No.: US 2009/0312621 A1
(43) Pub. Date: Dec. 17, 2009

(54) METHOD AND DEVICE FOR MONITORING A PHYSIOLOGICAL PARAMETER

(75) Inventors: Evgeny Verbitskiy, Eindhoven (NL); Pieter Klaas De Bokx, Eindhoven (NL); Theodorus Jacobus Johannes Denteneer, Eindhoven (NL); Golo Von Basum, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/376,137

(22) PCT Filed: Jul. 26, 2007
(86) PCT No.: PCT/IB07/52971

§ 371 Date: Feb. 3, 2009

(30) Foreign Application Priority Data
Aug. 8, 2006 (EP) 06118605.2

(51) Int. Cl. A61B 5/145

(52) U.S. Cl. 600/365

(57) ABSTRACT

The present invention is related to a method and device for monitoring a physiological parameter, like the blood glucose level, using prediction of future evolution of the physiological parameter based on continuous traces. The present method and device can be employed as a decision support system for diabetic patients.

### FIELD OF THE INVENTION

The present invention relates to a method and device for monitoring a physiological parameter.

### BACKGROUND OF THE INVENTION

About two hundred million people suffer from diabetes mellitus. Diabetes is a chronic condition in which the body does not properly regulate or utilize the insulin, which results in abnormal (too high/too low) levels of glucose in the blood. A tight glycemic monitoring is desirable for these patients. Presently, methods for observing the glucose levels require, for example, the finger-stick blood sample, which is further processed by a glucose meter. But observation alone is not sufficient for glycemic control. Any diabetic decision support system should be able to predict future values of the blood glucose with a time horizon of 30 to 60 minutes as accurately as possible. Within the predicted time the patient can then take the appropriate actions, as for example food intake or insulin injection, so that theses actions can become effective in time.

There are several problems which must be resolved by a prediction method. First of all, the dynamics of glucose-insulin interaction is rather complicated. Even continuous measurement of the blood glucose provides only part of the actual information about the state of the system. Secondly, fluctuations in blood glucose levels are intrinsically non-stationary and depend on external factors like food and insulin injections. Thirdly, even for the same patient, given the same food intake and insulin injections, the glucose dynamics can be different due to variation in a large number of parameters, as for example body temperature or physical activity, which are known to affect the glucose metabolic rates.

For example, in U.S. Pat. No. 6,923,763 B1 a prediction method is proposed based on a complex dynamic model that includes measured glucose values, heart rate and heart rate variability to try to estimate the blood glucose level in the future and estimate the risk of hypoglycemia. The approach is based on dynamic modeling of the glucose-regulation. One drawback of the prediction by dynamical models for glucose evolution is that the models are non-linear, complex and contain a large number of physiological parameters which cannot be measured.

### SUMMARY OF THE INVENTION

It is therefor an object of the present invention to provide a method for monitoring a physiological parameter with a more reliable prediction of the evolution of the physiological parameter.

The above objective is accomplished by a method for monitoring a physiological parameter, in particular for monitoring a blood glucose level, comprising
logging the physiological parameter by repeated measurements and
predicting the evolution of the physiological parameter,
wherein the evolution of the physiological parameter over an interval of autonomous evolution is predictable by a parametric model.

The logging comprises preferably a non-invasive, continuous method of measuring the physiological parameter, e.g. for glucose measurements. Continuous in this sense means, that, if required, the measurements are performed about every 5 to 10 minutes, preferably automatically.

An interval of autonomous evolution in the sense of this invention is meant to be a time interval with a relatively simple evolution of the physiological parameter, e.g. the blood glucose level. Such intervals are primarily characterized by the absence of drastic external influences like food intake, insulin injections, physical exercises, etc. Within an interval of autonomous evolution one is able to use a parametric model for the physiological parameter, like glucose concentration. The person skilled in the art understands that the parametric model can be derived from statistical analysis of continuous traces of the evolution of the physiological parameter, which can be described well approximated over a limited time interval. An advantage of the inventive method is, that a limited number of parameters and models of reduced complexity can be used, despite the complexity of underlying processes.

Within an autonomous interval, the evolution of the physiological parameter is predictable by the following parametric model:

G(t)=Q(t; p₁, . . . , pₖ),

where G(t) is the level of the physiological parameter at time t, which is predicted by the value Q at time t and with the parameters p₁, . . . , pₖ. The parametric model is used to extrapolate, i.e., predict the future values of the physiological parameter, especially the blood glucose concentration.

For the interval of autonomous evolution, which comprises a plurality of previous measurements (at times tₖ₋ᵢ, for i=1 . . . m), an optimal set of parameters or parameter vector \vec{p}ₖ₋₁ according to the parametric model is preferably determined by the Least Squares Method:

$${\vec{p}}_{k - 1} = {argmin}_{\vec{p} } {\sum }_{i = 1}^{m} {\vert G ( {t}_{k - i} ) - Q ( {t}_{k - i} ; \vec{p} ) \vert}^{2}$$

The prediction according to the inventive method comprises the steps of
adding a new measurement of the physiological parameter to the previous measurements of the interval of autonomous evolution,
conducting a test, whether the new measurement together with the previous measurements still form an interval of autonomous evolution, and depending upon this test,
adapting the interval of autonomous evolution and/or adapting the parametric model and
predicting the evolution of the physiological parameter, using the parametric model for the adapted interval of autonomous evolution.

A new measurement (tₖ, G(tₖ)) and a multitude of previous measurements of the interval of autonomous evolution {(tₖ₋ᵢ, G(tₖ₋ᵢ))}, with i running from 1 to m, together form a new interval upon which the test is conducted. The person skilled in the art understands, that in this test, the new measurement is compared to the previously predicted evolution of the physiological parameter. The interval of autonomous evolution is then adapted accordingly. For example the interval of autonomous evolution is narrowed such that the parametric model is able to predict the evolution of the physiological parameter, i.e. to predict the new measurement. The person skilled in the art also understands that the modified interval of autonomous evolution comprises at least the new measurement. When the interval of autonomous evolution is adapted, the parametric model is preferably adapted as well. The adapted parametric model is then used for the prediction of the future evolution of the physiological parameter.

The future evolution, using the optimal set of parameters, is predicted by the parametric model:

Ĝ(tₖ+Δt)=Q(tₖ+Δt; \vec{p}ₖ),

where Δt is the desired time horizon of 30 to 60 minutes.

Advantageously, the inventive parametric model, in principle, is not physical, i.e. the parametric model is primarily selected on the basis of adequate description of glucose traces, and not necessarily using complex models describing the full glucose regulatory system. Nevertheless, incorporation of at least parts of physical models is not excluded by the present invention. It is beneficial to use non-physical models, because they are simpler and contain fewer or no unknown parameters. For a complex non-linear dynamical system, which does not admit an analytic solution, proper estimation of the unknown parameters is much more difficult, if not infeasible. This can be done for type 1 diabetes, which is easier to model than type 2 or type 3 diabetes. The inventive method is advantageously applicable to glucose monitoring and prediction in general, and does not assume a particular type of diabetes.

The interval of autonomous evolution is preferably adapted by removing at least one measurement of the plurality of measurements of the interval of autonomous evolution. The person skilled in the art understands, that the oldest measurements of the interval of autonomous evolution are preferably removed first. The advantage of this method is, that an interval of autonomous evolution is not terminated, once the test is negative, but prediction is continued with a modified, smaller interval of autonomous evolution.

In a preferred embodiment of the present invention the test is conducted again, now testing whether the new measurement together with the measurements of the adapted interval form an interval of autonomous evolution. Furthermore preferred, the steps of removing at least one measurement and conducting the test is repeated until the adapted interval is an interval of autonomous evolution.

A preferred way of obtaining a new interval of autonomous evolution is, to repeatedly remove only the oldest measurement and then conduct the test, so that the longest possible, new interval of autonomous evolution can be determined. If, however, the plurality of measurements fails the test until only the new measurement is left, no interval of autonomous evolution can be determined. According to the inventive method, a new interval of autonomous evolution can be determined earliest after the next measurement.

A number of quantitative characteristics can be used to evaluate the quality of the new interval of autonomous evolution. Preferably, in the test an error is calculated by comparing the physiological parameter of the new measurement to a predicted physiological parameter of a preceding iteration:

εₖ=|G(tₖ)−Q(tₖ; \vec{p}ₖ₋₁)|

If the error does not exceed a predefined threshold, the interval of autonomous evolution is extended by adding a new observation value.

In another preferred embodiment, a new set of parameters for the parametric model is determined, wherein the parametric model with the new set of parameters optimally describes the evolution of the physiological parameter over the interval of autonomous evolution including the new measurement.

The new set of parameters is preferably determined after the Least Squares Method:

$${\vec{p}}_{k} = {argmin}_{\vec{p} } {\sum }_{i = 0}^{m} {\vert G ( {t}_{k - i} ) - Q ( {t}_{k - i} ; \vec{p} ) \vert}^{2}$$

Then, a distance between a new vector, defined by the new set of parameters and a former vector, defined by the set of parameters of the preceding iteration is calculated:

$${‖ {\vec{p}}_{k} - {\vec{p}}_{k - 1} ‖}^{2} = {\sum }_{i = 1}^{m} {\vert {p}_{k , i} - {p}_{k - 1 , i} \vert}^{2}$$

Again, if the distance does not exceed a predefined threshold value, the new measurement is added and the current interval of autonomous evolution is extended.

If the measurement is rejected in the test, the current interval of autonomous evolution is adapted. Most advantageously the new measurement can be added to the plurality of measurements forming the interval of autonomous evolution on the right, and the oldest measurements can be removed from the left until a smaller plurality of measurements meets the criterion of autonomous evolution.

In a preferred embodiment of the inventive method, a step of alarming a patient in case the predicted evolution of the physiological parameter is below or above a dangerous level. Advantageously, a patient suffering from diabetes can be alarmed early enough to prevent hypoglycemia or hyperglycemia.

Alternatively or additionally, an advice for the patient is provided, suggesting an agent or an amount of an agent to be administered, in particular an amount of insulin. The inventive system advantageously provides a decision support to the patient.

In a further preferred embodiment, a likelihood value is associated to the predicted evolution of the physiological parameter, the prediction of the evolution of the physiological parameter being discarded if the likelihood value is below a certain, predefined threshold value. The likelihood value advantageously reflects the trust in the accuracy of the prediction. For example, if the interval of autonomous evolution used for the prediction of glucose concentration is too small (too short), or, the interval was just adapted, this reduces the likelihood value. On the other hand, if the interval of autonomous evolution is relatively long and the set of optimal parameters has not changed drastically in a few last iterations, the likelihood value is higher. An advantage of the embodiment is to avoid prediction at all if the likelihood value is too small. The primary goal of the inventive monitoring and prediction of, for example, glucose concentrations is to prevent hypoglycemic and hyperglycemic events, i.e. events where glucose levels become dangerously low or dangerously high. These events typically occur at the end of long intervals of autonomous evolution. Hence, according to the present invention such predictions will have high likelihood values. Advantageously, extremely low or high glucose predictions cause an alarm for the patient and call for an appropriate action and a large number of false alarms is prevented.

An important aspect of the prediction is the stability of the results. Variations in the predicted evolution, which are faster than typical variations of glucose concentrations, should be avoided. In a preferred embodiment of the invention, the step of predicting the evolution of the physiological parameter is conducted using a multitude of parametric models for the adapted interval of autonomous evolution and obtaining the prediction as a weighted sum of the predictions of the multitude of parametric models:

$$Prediction ( t + Δ t ) = {\sum }_{j = 1}^{m} {w}_{j} Pred \cdot {Model}_{j} ( t + Δ t )$$

wherein the weights wⱼ of the parametric models are selected adaptively, preferably depending on a quality of the prediction of each parametric model in the previous iterations and/or depending on the number of iterations each parametric model has been used. For example, the weight wⱼ for the j-th parametric model reflects the quality of the prediction of the j-th model demonstrated so far and/or the time period the model during which the model has been used. If the weight of a particular parametric model drops below a certain predefined threshold value the parametric model is preferably discarded (or its weight is set to zero) and a substitute parametric model is added to the multitude of parametric models. One of the advantages of this embodiment is to increase the stability of the prediction. Machine Learning theory provides methods for collecting predictions from several models (“experts”) in one super-model (“super-expert”), which can outperform each of the individual “experts”.

Preferably a number of threshold values, particularly the threshold value for the weight of the parametric models and/or the threshold for the likelihood value are adapted. Advantageously, the variability between different individuals is met by this embodiment. Again, Machine Learning techniques may advantageously be used, to adapt the threshold values.

In a further preferred embodiment the inventive method comprises the step of adapting the interval of autonomous evolution and/or the parametric model depending on external parameters known to be affecting the physiological parameter. The external parameters are preferably associated to ingestion, administration of insulin, physical activity and/or emotional stress of the patient. These major factors, which influence the intervals of autonomous evolution in glucose dynamics, are preferably detected and the parametric model is adapted appropriately. This advantageously enhances the accuracy of the predicted evolution, as the intervals of autonomous evolution can be updated earlier, for example.

In a still further preferred embodiment, the external parameters are manually inputted by a patient or by an auxiliary. For example, a device can be equipped with “food”, “insulin”, “exercise” buttons, which will advantageously provide information on forthcoming changes in the interval of autonomous evolution. Alternatively calorie input forms, food lists and/or databases can be used.

According to another preferred embodiment at least one of the external parameters is detected by at least one sensor. Sensors could be heart rate sensors, heart rate variability sensors, insulin pump sensors or food intake sensors. The reliability of the prediction is advantageously enhanced, as it does not depend on the accurateness of the input of the patient, who may forget to give or give wrong input on food intake, exercise or amounts of injected insulin.

In a preferred embodiment the physical activity is determined by an accelerometer, more preferably by an accelerometer summarizing the acceleration in three directions in space. In a further preferred embodiment the physical activity is determined by analyzing a heart rate of a patient by a heart rate sensor. With relation to exercise, these embodiments advantageously provide relatively accurate readings for a parameter which is difficult to quantify for a patient.

Another aspect of the present invention is a monitoring device for monitoring a physiological parameter of a patient, in particular for monitoring a blood glucose level, comprising a physiological parameter measuring means and a computing means, the computing means having a data storage for storing measurements and a processor unit for calculating a prediction of the evolution of the physiological parameter, using the method according to the present invention. The monitoring device advantageously provides reliable prediction of the evolution of the physiological parameter.

The monitoring device preferably comprises input means for inputting external parameters which are known to affect the physiological parameter, such as ingestion, administration of insulin, physical activity and/or emotional stress of the patient. The input means preferably comprises at least a button for manual input of each external parameter. In this embodiment the quality of the predicted evolution is enhanced, as information on forthcoming changes in the interval of autonomous evolution is provided by the patient.

Preferably the monitoring device comprises output means for outputting alarm messages and/or advice messages to the patient. The inventive monitoring device thus provides a (diabetic) decision support system with the ability to predict future evolution of the physiological parameter, especially the blood glucose level, with a time horizon of 30 to 60 minutes.

Still another aspect of the present application is a monitoring device for a physiological parameter of a patient, in particular for monitoring a blood glucose level, comprising a physiological parameter measuring means and a computing means, the computing means having a data storage for storing the blood glucose measurements and a processor unit for calculating a prediction of the evolution of the physiological parameter, the monitoring device being connected to a detector for detecting a physical activity of the patient, preferably a three-axial accelerometer.

It is known that food intake, insulin uptake and exercise influence the blood glucose level most. The reliability of the prediction of the monitoring device usually depends on the accurateness of the input of the user. If he or she forgets to give or gives wrong input on food intake, exercise or amounts of injected insulin the model/device will give false predictions. With relation to exercise it is difficult anyhow to quantify the input. The monitoring device according to the present invention overcomes the abovementioned disadvantages by providing a monitoring device that comprises an detector for detecting a physical activity of the patient. This activity sensor senses the motions of a body and therewith provides a continuous input with relation to the physical activity of a person suffering from diabetes. In this way the input with regard to the exercise level is advantageously reliable.

The detector is preferably wearable by the patient, more preferably the detector is arranged in or at a belt, a watch or a mobile phone. This makes it easy to wear the detector all day long, which advantageously provides objective and detailed information on the duration and intensity of the activity of the patient (sleep, rest, sitting, exercise etc).

Preferably, the monitoring device in combination with the detector is initialized once to learn the specific reaction of a patient's activity on his or her blood glucose level, also in relation to prior food- and insulin-intake. Preferably, a first time usage requires an individual initialization of the model. For example, the user wears the detector together with the monitoring device and performs a set of standardized activities of different intensities. The prediction of the evolution of the physiological parameter is adapted during this initialization phase on an individual basis of the response of the different activity levels on the physiological parameter, in particular the blood glucose level. This information is used to update the parameter prediction model. An existing blood glucose level prediction device can be extended with the detector to improve the reliability of the blood glucose level evolution prediction.

### BRIEF DESCRIPTION OF THE INVENTION

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

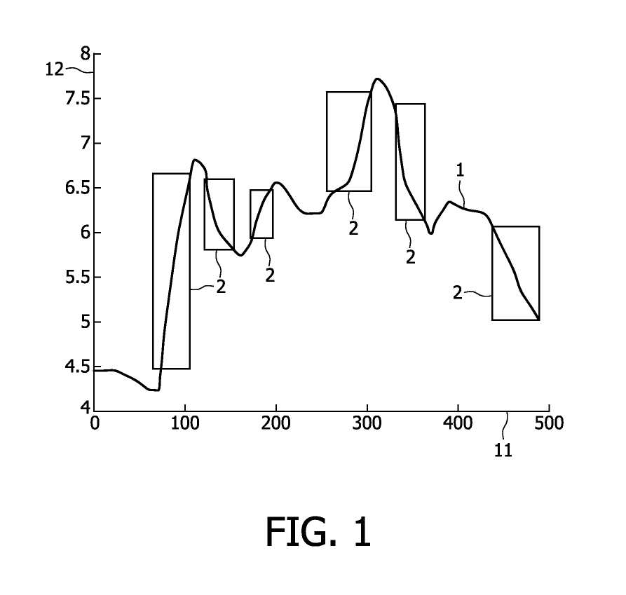

FIG. 1 depicts exemplarily a blood glucose evolution over an interval of time.

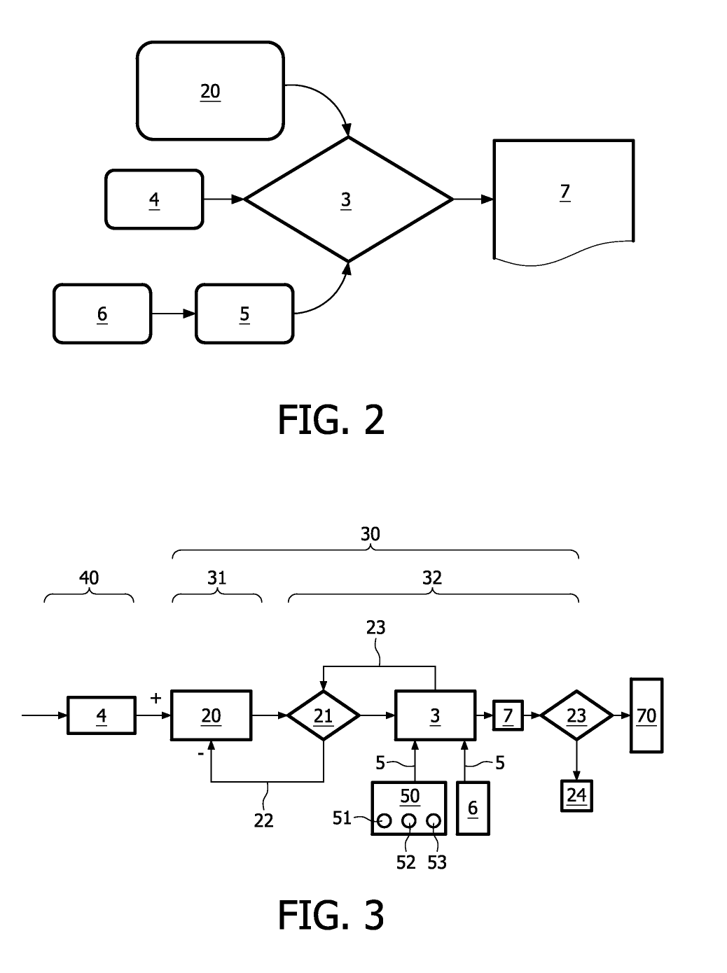

FIG. 2 illustrates schematically a method according to the present invention.

FIG. 3 illustrates schematically the method and embodiments of the monitoring device according to the present invention.

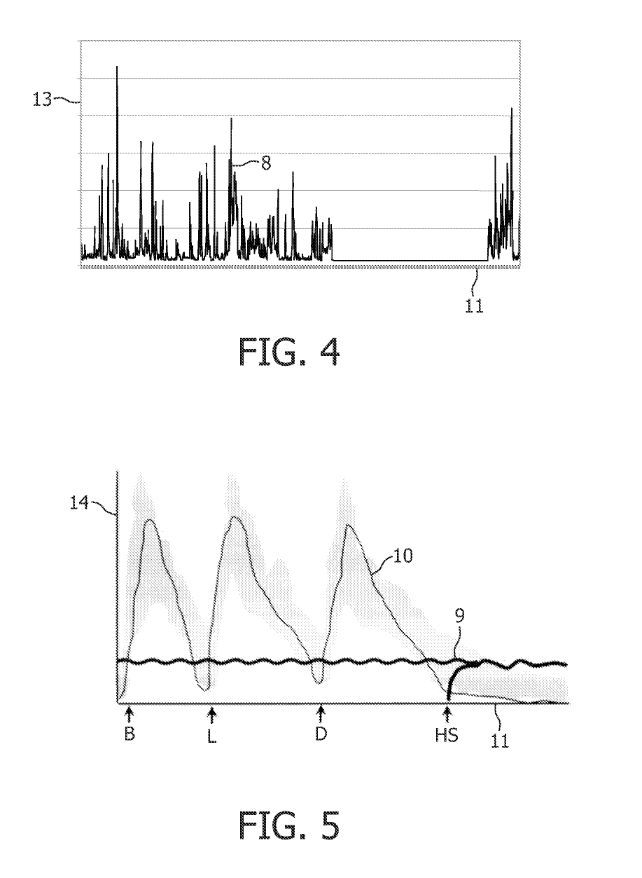

FIG. 4 depicts exemplarily a diagram of physical activity over an interval of time.

FIG. 5 illustrates an idealized pattern of insulin secretion over an interval of time.

### DETAILED DESCRIPTION OF THE EMBODIMENT

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

Fundamental work on glucoregulatory systems demonstrate without a doubt a great variation of possible dynamic evolutions of the blood glucose level 1, one of which is exemplarily depicted in FIG. 1. The abscissa 11 shows an interval of time in minutes and the axis of ordinates 12 the blood glucose concentration in milligrams per deciliter. Statistical analysis of continuous glucose traces 1 allows, that despite the complexity of underlying processes, glucose traces 1, limited in time, can be described and well approximated, using a limited number of parameters and models of reduced complexity. By inspecting glucose trace 1, it is possible to identify time intervals 2 with a relatively simple glucose evolution. Such time intervals 2 are referred to in here as intervals of autonomous evolution 2. Such intervals are primarily characterized by an absence of drastic external influences like food intake, insulin injections, physical exercise. Within an interval of autonomous evolution 2 it is possible to use a relatively simple parametric model for the glucose concentration.

In FIG. 2 the method according to the invention is depicted schematically. By analyzing a multitude of measurements, a parametric model 3 is created, which is able give a predicted evolution 7 for the physiological parameter with sufficient accuracy over an interval of autonomous evolution 2. Besides a new measurement 4 and stored previous measurements 20, no physical parameters are necessary for the inventive method. In order to enhance the prediction 7, the parametric model can incorporate external parameters 5, which are known to influence the physiological parameter. For example ingestion, insulin administration, physical activity or mental stress radically influence the blood glucose level. An external parameter 5 can be inputted manually or automatically by a sensor or detector 6, as for example a detector for the physical activity, such as a heart rate sensor or an accelerometer.

In FIG. 3 an embodiment of the inventive method is schematically depicted, further showing the appropriate parts of the inventive monitoring device. A measurement device 40 is used to take the new measurement 4, which is added to a plurality of former measurements, stored on a data storage 31. The plurality of former measurements represent the former interval of autonomous evolution 2 at the time of a previous measurement. The new measurement 4 and the plurality of former measurements form together a new plurality of measurements 20. A test 21 is conducted, whether the new plurality of measurements 20 forms an interval of autonomous evolution. For the test 21, the former prediction of the future evolution by the parametric model 3 is compared to the new measurement 4, which is shown by feedback line 23. If the criterion of an interval of autonomous evolution is not met, at least one, preferably the oldest measurement 22 of the plurality of measurements 20 is removed and the test 21 is repeated. If the criterion of an interval of autonomous evolution is met, the parametric model 3 is adapted if necessary and a prediction 7 for the future evolution of the physiological parameter is calculated. A likelihood value 23, representing the reliability of the predicted evolution 7, is also calculated. If the likelihood value 23 is below a predefined threshold, the prediction 7 is discarded 24. Otherwise the predicted evolution 7 is outputted by an output means 70. The process starts again upon the next new measurement. All mentioned calculations are performed by a processor unit 32, which is, like data storage 31, part of a computing means 30.

According to an embodiment of the invention, the parametric model 3 is additionally adapted depending upon external parameters 5, which are derived from an input means 50 and/or from a detector 6. The input means 50 is preferably equipped with buttons 51, 52, 53, which are pressed by a patient upon certain activities in order to provide information on forthcoming changes in the interval of autonomous evolution to the monitoring device. For example button 51 is used for ingestion, button 52 for insulin administration and button 53 for physical exercises. Detector 6 preferably detects physical activity of the patient automatically, for example by way of a three-axial accelerometer worn by the patient.

FIG. 4 shows a diagram of a detected activity pattern 8 for a person who wore the detector 6 for 24 hours. The time is shown on the abscissa 11. For example a tri-axial accelerometer gives every minute the sum of accelerations in three directions. These accelerations are shown on the axis of ordinates. They can be converted into a calorific value, but the arbitrary units can as well be directly used by the parametric model.

FIG. 5 shows an idealized pattern of insulin secretion for a healthy individual who has consumed three standard meals, breakfast B, lunch L and dinner D. Again on the abscissa 11 the time is shown. On the ordinate the insulin effect is qualitatively shown. HS marks the bedtime. People with diabetes can use two types of insulin; the long acting insulin or basal insulin 9 and the short acting insulin 10 (bolus). The bolus type of insulin 9 is mostly injected before the onset of a meal. The effects of the long acting insulin 10 can be predicted more accurately by the inventive method and monitoring device. A day of less activity or of higher activity than normal, influences the insulin level, which may result in hyperglycemia or hypoglycemia. The detector for detecting physical activity measures and summarizes the acceleration, compares it to a day of normal activity and the monitoring system can warn the patient that he/she is more/less active and should adapt his insulin profile.

The present invention is related to a method and device for monitoring a physiological parameter, like the blood glucose level, using prediction of future evolution of the physiological parameter based on continuous traces. The present method and device can be employed as a decision support system for diabetic patients.

1. Method for monitoring a physiological parameter (1), in particular for monitoring a blood glucose level, comprising
logging the physiological parameter (1) by repeated measurements (4) and
predicting the evolution of the physiological parameter (1),
wherein the evolution of the physiological parameter over an interval of autonomous evolution (2) is predictable by a parametric model (3), the prediction comprising the steps of
adding a new measurement (4) of the physiological parameter to the previous measurements (20) of the interval of autonomous evolution (2),
conducting a test (21), whether the new measurement together with the previous measurements still form an interval of autonomous evolution and, depending upon this test,
adapting the interval of autonomous evolution and/or adapting the parametric model (3) and
predicting (7) the evolution of the physiological parameter, using the parametric model (3) for the adapted interval of autonomous evolution.

2. Method according to claim 1, wherein the interval of autonomous evolution (2) is adapted by removing at least one measurement (22) of a plurality of measurements of the interval of autonomous evolution.

3. Method according to claim 2, wherein the test is conducted, whether the new measurement together with the measurements of the adapted interval form an interval of autonomous evolution.

4. Method according to claim 3, wherein the steps of removing at least one measurement (22) and conducting the test is repeated until the adapted interval is an interval of autonomous evolution.

5. Method according to claim 1, wherein the step of conducting the test comprises
calculation of an error by comparing the physiological parameter of the new measurement to a predicted physiological parameter of a preceding iteration, or
determination of a new set of parameters for the parametric model, wherein the parametric model with the new set of parameters optimally describes the evolution of the physiological parameter over the interval of autonomous evolution including the new measurement and calculation of a distance between a new vector, defined by the new set of parameters and a former vector, defined by a set of parameters of a preceding iteration.

6. Method according to claim 1, further comprising
the step of alarming a patient in case the predicted evolution (7) of the physiological parameter (1) is below or above a dangerous level and/or
the step of outputting an advice, in particular suggesting an agent or an amount of an agent to be administered, in particular an amount of insulin.

7. Method according to claim 1, further associating a likelihood value to the predicted evolution of the physiological parameter, the prediction of the evolution of the physiological parameter being discarded (23) if the likelihood value is below a certain threshold value.

8. Method according to claim 1, the step of predicting the evolution of the physiological parameter is conducted using a multitude of parametric models for the adapted interval of autonomous evolution and obtaining the prediction as a weighted sum of the predictions of the multitude of parametric models, wherein the weights of the parametric models are defined adaptively, preferably depending upon a quality of the prediction of each parametric model in the previous iterations and/or depending upon the number of iterations each parametric model has been used.

9. Method according to claim 8, wherein each parametric model is discarded when its weight drops below a threshold value and a substitute parametric model is added to the multitude of parametric models.

10. Method according to claim 8, wherein a number of threshold values, particularly the threshold value for the weight of the parametric models and/or the threshold for the likelihood value are adapted.

11. Method according to claim 1, further comprising the step of adapting the interval of autonomous evolution (2) and/or the parametric model (3) depending on external parameters (5) known to be affecting the physiological parameter (1).

12. Method according to claim 11, wherein the external parameters (5) are associated to ingestion, administration of insulin, physical activity and/or emotional stress of the patient.

13. Monitoring device for monitoring a physiological parameter (1) of a patient, in particular for monitoring a blood glucose level, comprising a physiological parameter measuring means (40) and a computing means (30), the computing means having a data storage (31) for storing measurements (4) and a processor unit (32) for calculating a prediction of the evolution of the physiological parameter, according to the method of claim 1.

14. Monitoring device according to claim 13, further comprising input means (50) for inputting external parameters (5) which are known to affect the physiological parameter (1), such as ingestion, administration of insulin, physical activity and/or emotional stress of the patient.

15. Monitoring device according to claim 14, wherein the input means comprises at least one button (51, 52, 53) for manual input of each external parameter.

16. Monitoring device according to claim 13, further comprising output means (70) for outputting alarm messages and/or advice messages to the patient.

17. Monitoring device for a physiological parameter (1) of a patient, in particular for monitoring a blood glucose level, comprising a physiological parameter measuring means (40) and a computing means (30), the computing means having a data storage (31) for storing the measurements (4) and a processor unit (32) for calculating a prediction of the evolution of the physiological parameter, the monitoring device being connected to a detector (6) for detecting a physical activity of the patient, preferably a three-axial accelerometer.
